(19) United States
(12) Patent Application Publication
(10) Pub. No.: US 2006/0195116 A1
(43) Pub. Date: Aug. 31, 2006

(54) D-Tail patellar bone tunneling system

(76) Inventor: Michael D. Fox, Cedar Park, TX (US)

Correspondence Address: STURM & FIX LLP, 206 SIXTH AVENUE, SUITE 1213, DES MOINES, IA, 50309-4076, US

(21) Appl. No.: 11/362,301

(22) Filed: Feb. 24, 2006

Related U.S. Application Data
(60) Provisional application No. 60/656,574, filed on Feb. 25, 2005.

(51) Int. Cl. A61B 17/60

(52) U.S. Cl. 606/96

(57) ABSTRACT

An apparatus used to create bone tunnels for a repair of a patellar tendon rupture, the apparatus comprising a base having an upper surface and a lower surface which has a plurality of orifices passing through the base, a plurality of grooves, at least one for each orifice, each of the plurality of grooves to provide passage between the respective orifice and the lower surface of the base, a receiving post operatively, rigidly attached to the upper surface of the base, a locking lever, operatively, pivotally attached to the receiving post, an aperture passing through the receiving post in an orientation parallel to the plurality of grooves, and a retaining guide. The retaining guide has a locking bar operatively, removably, and slidably engaged in the aperture and locked by the locking lever, and an alignment post.

### CROSS-REFERENCE TO RELATED APPLICATIONS

This application contains disclosure from and claims the benefit under Title 35, United States Code, §119(e) of the following U.S. Provisional Application: U.S. Provisional Application Ser. No. 60/656,574 filed Feb. 25, 2005, entitled D-TAIL PATELLAR BONE TUNNELING SYSTEM.

### STATEMENT REGARDING FEDERALLY SPONSORED RESEARCH OR DEVELOPMENT

Not Applicable

### BRIEF DESCRIPTION OF THE SEVERAL VIEWS OF THE DRAWINGS

FIG. 1 is a vertical view of the receiving end of the bone tunneling tower;

FIG. 2 is a top view of the bone tunneling tower;

FIG. 3 is a view of the bone tunneling tower lying on its side;

FIG. 4 is the retaining guide viewed from the receiving end;

FIG. 5 is a top view of the retaining guide;

FIG. 6 is a view of the retaining guide lying on its side;

FIG. 7 is the D-Tail drill; and

FIG. 8 is a side view of the D-Tail drill showing the unique features of this drill bit.

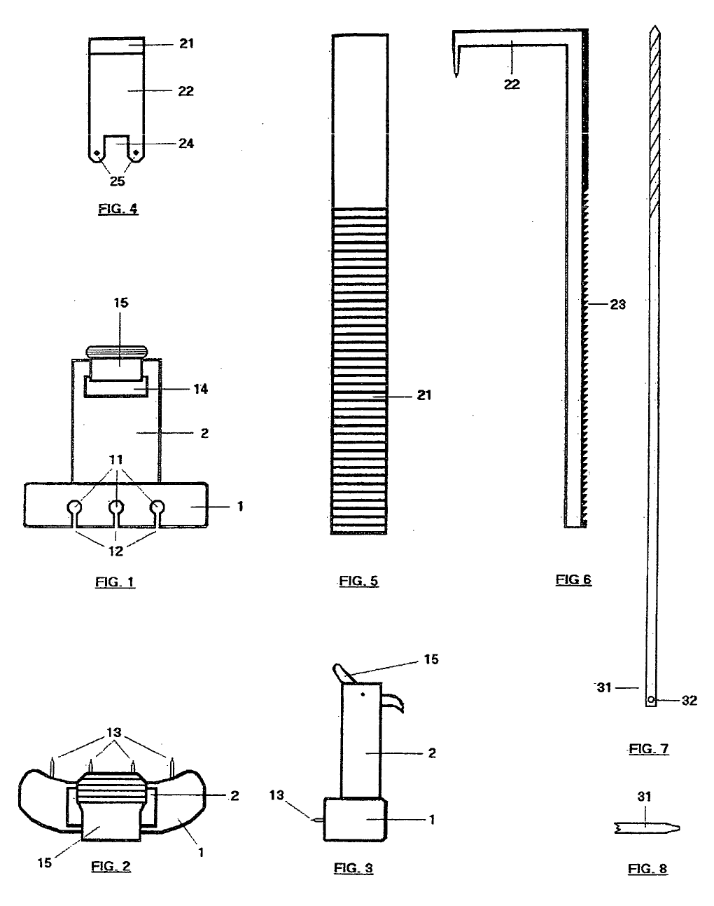

### DETAILED DESCRIPTION OF THE INVENTION

The bone tunneling tower is made up of the tower base (1) and the receiving post (2). The tower base has three drill guide holes (11) which receive the D-Tail drill. A groove (12) is cut out of all three guide holes at the bottom section to allow for suture release. On the exiting end of the tower base there are four pointed retaining pins (13) spaced equally between the guide holes. The receiving post has a window (14) in which the proximal end of the retaining guide enters. At the top of the receiving post there is a spring loaded locking lever (15) which locks the retaining guide at the required position.

The retaining guide is made up of a locking bar (21) and an alignment post (22). A plurality of locking grooves (23) encompasses the top of the locking bar for the locking lever to engage. The alignment post extends from the receiving end of the locking bar downward at a fixed length. At the end of the alignment post there is an exit window (24) in which the D-Tail drill bit passes through. On each side of the exit window is a pointed retaining pin (25).

The D-Tail drill is a three fluted drill bit of a determined length. The tail 31 is designed with a hole (32) of determined diameter for securing suture in preparation of passing the suture through the bone tunnels. The tail of the drill bit is tapered from a determined point and continues to the end of the drill as seen in FIG. 8.

In operation the surgeon uses this system to create the bone tunnels necessary and provide a means of suture passing for the repair of a patellar or quadriceps tendon rupture. Before the surgeon applies this system to the procedure, the approach and soft tissue preparation must first be performed in the usually fashion. After completion of the above mentioned, the surgeon assembles the drill guide by placing the retaining guide and bone tunneling tower together. The locking bar will slide into the window of the bone tunneling tower and begin to lock into the locking lever. At this point the surgeon will place the retaining pins of the retaining guide into the uninjured pole of the patella centered in the anterior posterior plane as well as in the medial lateral plane. Then the surgeon will continue locking the guide by squeezing the two components together until contact is made between the injured pole of the patella and the retaining pins of the bone tunneling tower. These pins should also be centered as explained above. After proper placement of all the pins have been established the surgeon must then squeeze the guide together a final time to engage all of the retaining pins deeper into the patella and continue the locking process. At this point, the drill guide is ready for the insertion of the drill bit. The tail end of the drill bit is placed into a pin driver of a surgical drill and the top of the drill bit is placed into the receiving end of the tower base through one of the three drill guide holes. Under the power of the drill, the drill bit is then pushed through the patella until a generous amount of the tip protrudes through the uninjured pole of the patella. The drill is separated from the drill bit and the suture is threaded through the hole in the tail of the drill bit. The drill bit is now ready for passing of the suture and should be grabbed from the tip and pulled out of the patella from the uninjured pole using the surgeons' tool of choice. A pin puller should work well in this situation. This stop will pass the suture through the patellar tunnel releasing it on the proper end for securing. This will be repeated two more times through the remaining holes and the drill guide will be ready for removal. Upon removal of the drill guide the locking lever must first be pressed releasing the retaining guide from the bone tunneling tower. The two components must be separated completely leaving the bone tunneling tower in place. The suture must now be drawn through the grooves at the bottom of the guide holes in order to withdraw the bone tunneling tower from the surgical field. The surgeon may continue the surgical procedure in the usual fashion to complete the repair of a patellar or quadriceps tendon rupture.

We claim:

1. An apparatus used to create bone tunnels for a repair of a patellar tendon rupture, the apparatus comprising:
a base having an upper surface and a lower surface;
a plurality of orifices passing through the base;
a plurality of grooves, at least one for each orifice, each of said plurality of grooves to provide passage between the respective orifice and the lower surface of the base;
a receiving post operatively, rigidly attached to the upper surface of the base;
a locking lever, operatively, pivotally attached to the receiving post;
an aperture passing through the receiving post in an orientation parallel to the plurality of grooves; and
a retaining guide, said retaining guide comprising a locking bar operatively, removably, and slidably engaged in the aperture and locked by the locking lever, and an alignment post.

2. The apparatus of claim 1 additionally comprising:
a drill bit having a predetermined length and a first end and a second end;
a plurality of flutes at the first end of the drill bit;
an eye passing diametrically through the second end of the drill bit; and
sutures engagable in the eye of the drill bit.
